(19) United States
(12) Patent Application Publication
(10) Pub. No.: US 2019/0117198 A1
(43) Pub. Date: Apr. 25, 2019

(54) THREE-DIMENSIONAL RECONSTRUCTION METHOD, APPARATUS AND DEVICE AND STORAGE MEDIUM FOR CORONARY VESSELS

(71) Applicant: SHENZHEN UNIVERSITY, SHENZHEN (CN)

(72) Inventors: Yong ZHANG, SHENZHEN (CN); Xiansong WU, SHENZHEN (CN); Heye ZHANG, SHENZHEN (CN); Zhifan GAO, SHENZHEN (CN); Xin LIU, SHENZHEN (CN)

(73) Assignee: SHENZHEN UNIVERSITY

(21) Appl. No.: 16/166,117

(22) Filed: Oct. 21, 2018

Related U.S. Application Data
(63) Continuation of application No. PCT/CN2017/091573, filed on Jul. 4, 2017.

(30) Foreign Application Priority Data
Jun. 30, 2017 (CN) 201710526406.7

(51) Int. Cl. A61B 8/08; G06T 7/12; G06T 7/149; G06T 17/00

(52) U.S. Cl. CPC A61B 8/5269; G06T 7/12; G06T 2207/30048; A61B 8/483; G06T 17/00; G06T 7/149

(57) ABSTRACT

A three-dimensional reconstruction method for coronary vessels includes: pretreating a coronary angiography (CAG) image, extracting a vessel edge profile and two-dimensional guide wires, and dividing inner and outer membranes for intravascular ultrasound (IVUS) images; translating the two-dimensional guide wires located in a first angiography plane and a second angiography plane in the CAG image to the same starting point; building perpendicularly intersected curved surfaces; setting an intersecting line as a three-dimensional guide wire; arranging the IVUS images per frame at equal intervals along the three-dimensional guide wire; rotating the IVUS images to positions perpendicular to tangent vectors in corresponding positions; rotating the IVUS images in a vertical plane of the tangent vectors; back projecting the IVUS images to the CAG image; determining optimal orientation angles according to distances from the back projections and the vessel edge profile to the three-dimensional guide wire; and finally reconstructing a vessel surface.

### CROSS-REFERENCE TO RELATED APPLICATIONS

This application is a continuation of International Patent Application No. PCT/CN2017/091573 with a filing date of Jul. 4, 2017, designating the United States, now pending, and further claims priority to Chinese Patent Application No. 201710526406.7 with a filing date of Jun. 30, 2017. The content of the aforementioned applications, including any intervening amendments thereto, are incorporated herein by reference.

### TECHNICAL FIELD

The present disclosure belongs to the technical field of computers, and particularly relates to a three-dimensional reconstruction method, apparatus, device and a storage medium for coronary vessels.

### BACKGROUND OF THE PRESENT INVENTION

The morbidity of coronary heart disease and the mortality of patients are increasing in recent years. The main clinical diagnosis of the coronary heart disease includes coronary angiography (CAG) and intravascular ultrasound (IVUS). CAG is the “gold standard” for the diagnosis of the coronary heart disease currently, and CAG can be used to determine whether there is stenosis in coronary arteries as well as the position, extent and range of the stenosis. IVUS can be used to obtain the shape and degree of stenosis of the vascular walls in coronary arteries. However, CAG images cannot provide structural information and lesion degree of the vascular walls, and IVUS cannot provide axial positions and spatial directions of vascular sections. To simultaneously examine shapes and structures, and intraluminal lesion information of the vessels, a technical means is needed to complement the respective advantages of CAG and IVUS in the display of the shapes of the coronary arteries, so as to truly reflect the anatomic structure and spatial geometry of the vessels.

At present, a method to realize complementary advantages of CAG and IVUS in the display of the shapes of the coronary arteries is mainly to realize three-dimensional reconstruction of guide wires based on a binocular imaging principle. The method has high requirements for known conditions of parameters. Most of clinical angiography images only record angiography angles of the angiography process, and do not record linear distances from an X-ray source to angiography planes. It is also possible that recorded parameters are lost, which brings a large error to three-dimensional reconstruction.

### SUMMARY OF PRESENT INVENTION

The purpose of the present disclosure is to provide a three-dimensional reconstruction method, apparatus, device and a storage medium for coronary vessels, so as to solve the problems of large errors and low accuracy in three-dimensional reconstruction of coronary vessels due to high requirements for known degree of parameters in a method for collecting image data of CAG and IVUS in the prior art.

In one aspect, the present disclosure provides a three-dimensional reconstruction method for coronary vessels, including the following steps: pretreating an inputted coronary angiography image, extracting a vessel edge profile and two-dimensional guide wires from the pretreated coronary angiography image, and dividing inner and outer membranes for inputted associated intravascular ultrasound images; translating the two-dimensional guide wires located in a first angiography plane and a second angiography plane in the coronary angiography image to the same starting point, building perpendicularly intersected curved surfaces according to the translated two-dimensional guide wires, and setting an intersecting line of the perpendicularly intersected curved surfaces as a three-dimensional guide wire; arranging the intravascular ultrasound images per frame at equal intervals along the three-dimensional guide wire, and rotating the intravascular ultrasound images to positions perpendicular to tangent vectors according to the tangent vectors in positions of the intravascular ultrasound images on the three-dimensional guide wire; rotating the intravascular ultrasound images in the corresponding positions of the tangent vectors at different angles in a vertical plane of the tangent vectors, back projecting the rotated intravascular ultrasound images to the coronary angiography image, and determining optimal orientation angles of the intravascular ultrasound images per frame according to distances from the back projections of the intravascular ultrasound images and the vessel edge profile to the three-dimensional guide wire; and rotating the intravascular ultrasound images per frame to the corresponding optimal orientation angles, and conducting surface reconstruction on vessels of the coronary angiography image and the intravascular ultrasound images according to a span difference between inner membranes and a span difference between outer membranes in the intravascular ultrasound images per frame on the three-dimensional guide wire.

In another aspect, the present disclosure provides a three-dimensional reconstruction apparatus for coronary vessels, including: an image treatment unit for pretreating an inputted coronary angiography image, extracting a vessel edge profile and two-dimensional guide wires from the pretreated coronary angiography image, and dividing inner and outer membranes for inputted associated intravascular ultrasound images; a guide wire reconstruction unit for translating the two-dimensional guide wires located in a first angiography plane and a second angiography plane in the coronary angiography image to the same starting point, building perpendicularly intersected curved surfaces according to the translated two-dimensional guide wires, and setting an intersecting line of the perpendicularly intersected curved surfaces as a three-dimensional guide wire; an ultrasound image positioning unit for arranging the intravascular ultrasound images per frame at equal intervals along the three-dimensional guide wire and rotating the intravascular ultrasound images to positions perpendicular to tangent vectors according to the tangent vectors in positions of the intravascular ultrasound images on the three-dimensional guide wire; an ultrasound image orientation unit for rotating the intravascular ultrasound images in the corresponding positions of the tangent vectors at different angles in a vertical plane of the tangent vectors, back projecting the rotated intravascular ultrasound images to the coronary angiography image, and determining optimal orientation angles of the intravascular ultrasound images per frame according to distances from the back projections of the intravascular ultrasound images and the vessel edge profile to the three-dimensional guide wire; and a surface reconstruction unit for rotating the intravascular ultrasound images per frame to the corresponding optimal orientation angles, and conducting surface reconstruction on vessels of the coronary angiography image and the intravascular ultrasound images according to a span difference between inner membranes and a span difference between outer membranes in the intravascular ultrasound images per frame on the three-dimensional guide wire.

In another aspect, the present disclosure also provides a medical device, including a memory, a processor and a computer program stored in the memory and executed on the processor, wherein the processor, when executing the computer program, realizes the steps of the three-dimensional reconstruction method for coronary vessels as mentioned above.

In another aspect, the present disclosure also provides a computer readable storage medium, storing the computer program, wherein the computer program, when executed by the processor, realizes the steps of the three-dimensional reconstruction method for coronary vessels as mentioned above.

The present disclosure includes: pretreating the coronary angiography image, extracting the vessel edge profile and the two-dimensional guide wires, dividing inner and outer membranes for the intravascular ultrasound images, translating the coronary angiography images located in a preset first angiography plane and a preset second angiography plane, building perpendicularly intersected curved surfaces according to the translated two-dimensional guide wires, setting an intersecting line of the curved surfaces as a three-dimensional guide wire, arranging the intravascular ultrasound images per frame at equal intervals along the three-dimensional guide wire, rotating the intravascular ultrasound images so that the intravascular ultrasound images are perpendicular to tangent vectors in corresponding positions of on the three-dimensional guide wire, rotating the corresponding intravascular ultrasound images at different angles in a vertical plane of the tangent vectors; back projecting the rotated intravascular ultrasound images to the coronary angiography image, determining optimal orientation angles of the intravascular ultrasound images per frame according to the distances from the back projections and the vessel edge profile to the three-dimensional guide wire, and conducting surface reconstruction on the vessels according to the span difference between inner membranes and the span difference between outer membranes in the intravascular ultrasound images per frame on the three-dimensional guide wire, thereby realizing the integration of the coronary angiography image and the intravascular ultrasound images, so that shapes and structures, and intraluminal lesion information of the vessels can be examined simultaneously. In addition, the present disclosure effectively reduces the influence of image noise on vascular reconstruction due to respiration of patients, effectively solves the influence caused by lack of parameters or incomplete parameter calibration in an angiography device, and effectively enhances efficiency and accuracy of three-dimensional reconstruction of the coronary vessels.

### DESCRIPTION OF THE DRAWINGS

FIG. 1 is a flow chart of a three-dimensional reconstruction method for coronary vessels according to embodiment 1 of the present disclosure;

FIG. 2 is a schematic diagram showing extraction of a vessel edge profile and a two-dimensional guide wire in the three-dimensional reconstruction method for coronary vessels according to embodiment 1 of the present disclosure;

FIG. 3 is a schematic diagram showing generation of a three-dimensional guide wire in the three-dimensional reconstruction method for coronary vessels according to embodiment 1 of the present disclosure;

FIG. 4 is a schematic diagram showing distances from a back projection of an intravascular ultrasound image and a vessel edge profile to a three-dimensional guide wire in the three-dimensional reconstruction method for coronary vessels according to embodiment 1 of the present disclosure;

FIG. 5 is a schematic diagram showing conducting surface reconstruction on vessels through target profile lines of an upper layer and a lower layer in the three-dimensional reconstruction method for coronary vessels according to embodiment 1 of the present disclosure;

FIG. 6 is a structural schematic diagram of a three-dimensional reconstruction apparatus for coronary vessels according to embodiment 2 of the present disclosure;

FIG. 7 is a preferred structural schematic diagram of the three-dimensional reconstruction apparatus for coronary vessels according to embodiment 2 of the present disclosure; and

FIG. 8 is a structural schematic diagram of a medical device according to embodiment 3 of the present disclosure.

### DETAILED DESCRIPTION OF PREFERRED EMBODIMENTS

To make the purpose, the technical solution and the advantages of the present disclosure more clear, the present disclosure is further described below in detail in combination with drawings and embodiments. It should be understood that specific embodiments described herein are merely used to explain the present disclosure, not to limit the present disclosure.

The specific implementation of the present disclosure is described below in detail in combination with specific embodiments.

### Embodiment 1

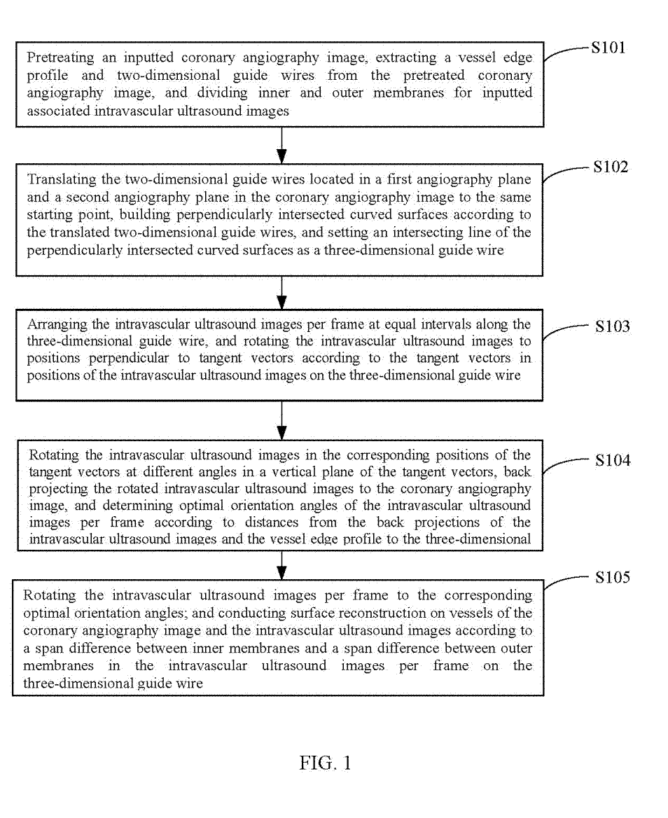

FIG. 1 shows a flow chart of a three-dimensional reconstruction method for coronary vessels according to embodiment 1 of the present disclosure. For the convenience of explanation, only the part related to the embodiments of the present disclosure is shown as follows:

In step S101, an inputted coronary angiography image is pretreated, a vessel edge profile and two-dimensional guide wires are extracted from the pretreated coronary angiography image, and inner and outer membranes are divided for inputted associated intravascular ultrasound images.

In the embodiment of the present disclosure, the inputted coronary angiography image and the corresponding or associated intravascular ultrasound images may be from a medical database provided by a hospital, wherein a coronary angiography device may record the coronary angiography image of a patient from multiple angles. The inputted coronary angiography image may be the coronary angiography images in any two directions. Herein, the two directions are called as a first angiography plane and a second angiography plane. The inputted intravascular ultrasound images are multi-frame vessel sectional images recorded when the guide wire is withdrawn at uniform speed from a far end of a target change position.

In the embodiment of the present disclosure, because the coronary angiography image is interfered by various factors during imaging, transmission and storage, it is easy to generate noise on the image. To treat the coronary angiography image more accurately, the coronary angiography image needs to be pretreated. In the pretreatment process, a filter can be used to map the coronary angiography image to a new image and enhance the contrast of the coronary angiography image (e.g., adjust a low intensity value in a preset percentage of the image intensity value to be lower, and adjust a high intensity value to be higher), so as to remove some pseudo images of the coronary angiography image. For example, thoracic bone, muscular tissue and other anatomic sites of the patient may be shown as vessels on a local angiography image, and the vessel profile and the guide wire in the coronary angiography image are extracted more conveniently. Because the noise of the coronary angiography image mainly includes Gaussian noise and salt-and-pepper noise, random noise and the salt-and-pepper noise in the coronary angiography image can be treated through a preset Gaussian low-pass filter.

In the embodiment of the present disclosure, the vessel edge profile and two-dimensional guide wires are extracted from the coronary angiography image; and the two-dimensional guide wires can be understood as vessel central lines in the coronary angiography image. The vessel edge profile can be extracted through a preset Gaussian-Laplace (LOG) operator, so as to smooth the vessel edge profile and eliminate the noise generated at the time of extracting the vessel edge profile. The two-dimensional guide wires can be extracted through a preset Hessian matrix. Specifically, second-order Taylor series expansion is conducted on the coronary angiography image to obtain the Hessian matrix of the coronary angiography image:

I(P+ΔP)≈I(P)+ΔP∇I(P)+ΔP^{T}H(P)ΔP,

wherein I is n-dimensional data and represents two-dimensional data herein, i.e., the coronary angiography image; P is a point in the two-dimensional image data ∇I(P) is a gradient vector of P point; and H(P) is the Hessian matrix of P point. The Hessian matrix of the coronary angiography image can be represented as:

$$H ( P ) = [ \begin{matrix}{I}_{xx} & {I}_{xy} \\ {I}_{yx} & {I}_{yy}\end{matrix} ] ,$$

wherein

$${I}_{xy} = \frac{\partial I}{\partial x \partial y} ;$$

Iₓₓ, I_{xy}, I_{yx} and I_{yy} are second-order differentials of the coronary angiography image and can be obtained by the convolution of the second derivative of the coronary angiography image and the Gaussian filter. A feature value with a larger absolute value of the Hessian matrix and a corresponding feature vector represent the intensity and the direction of P point at a large curvature; and a feature value with a smaller absolute value and a corresponding feature vector represent the intensity and the direction of P point at a small curvature. It can be seen that the feature vector corresponding to the feature value with a larger absolute value of the Hessian matrix of the coronary angiography image is perpendicular to a local vascular skeleton; the feature vector corresponding to the feature value with a smaller absolute value is parallel to the local vascular skeleton; and the two-dimensional guide wires can be extracted through the feature that the feature vector corresponding to the feature value with a smaller absolute value is parallel to the local vascular skeleton. After extraction, the image of the extracted two-dimensional guide wires can be corroded and refined to eliminate the interference perpendicular to the travel direction of the vessel, eliminate communication branch with a small area and conduct interpolation and fitting to obtain the guide wire curve of two-dimensional vessels, i.e., the two-dimensional guide wires, so that the accurate positions of the two-dimensional guide wires can be still found when the vessel is changed suddenly.

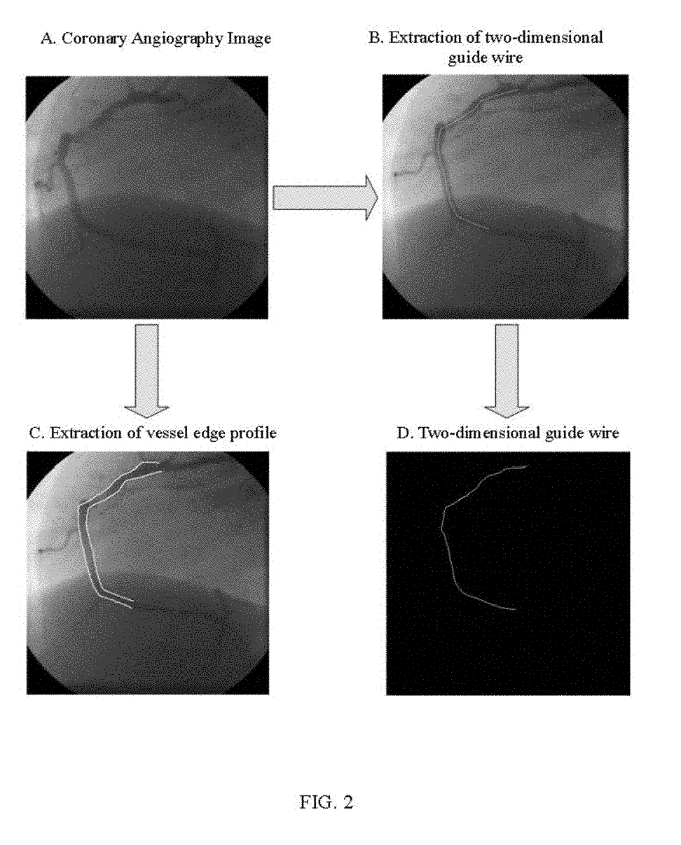

In the embodiment of the present disclosure, inner and outer membranes are divided for the intravascular ultrasound images; and inner and outer membranes are divided for the intravascular ultrasound images per frame through IVUS Angio tool software (public available software for treating intravascular images). The software can be combined with an electrocardiogram to identify an IVUS image at the end of diastole on the basis of R wave detection, to realize automatic division of inner and outer membranes. If the electrocardiogram is not provided simultaneously, the IVUS image at the end of diastole can be manually selected and manually corrected. As shown in FIG. 2, A to C in the figure indicates the extraction of the vessel edge profile, and A to B to D indicates the extraction of the two-dimensional guide wires.

In step S102, the two-dimensional guide wires located in a first angiography plane and a second angiography plane in the coronary angiography image are translated to the same starting point; perpendicularly intersected curved surfaces are built according to the translated two-dimensional guide wires; and an intersecting line of the perpendicularly intersected curved surfaces is set as a three-dimensional guide wire.

In the embodiment of the present disclosure, because the starting point of the guide wires is fixed, the two-dimensional guide wires in the coronary angiography image in different directions (the first angiography plane and the second angiography plane) are translated to the same starting point (or the same height). After translation, according to the two-dimensional guide wires, a first curved surface perpendicularly intersected with the coronary angiography image in the first angiography plane is built and a second curved surface perpendicularly intersected with the coronary angiography image in the second angiography plane is built. The first curved surface and the second curved surface are perpendicularly intersected, and the obtained intersecting line is set as a three-dimensional guide wire, i.e., a three-dimensional curve of the guide wires, thereby effectively reducing errors generated by the three-dimensional guide wire due to no calibration of part of parameters or parameter deviation in the angiography device and reducing geometric distortion caused by respiration of patients.

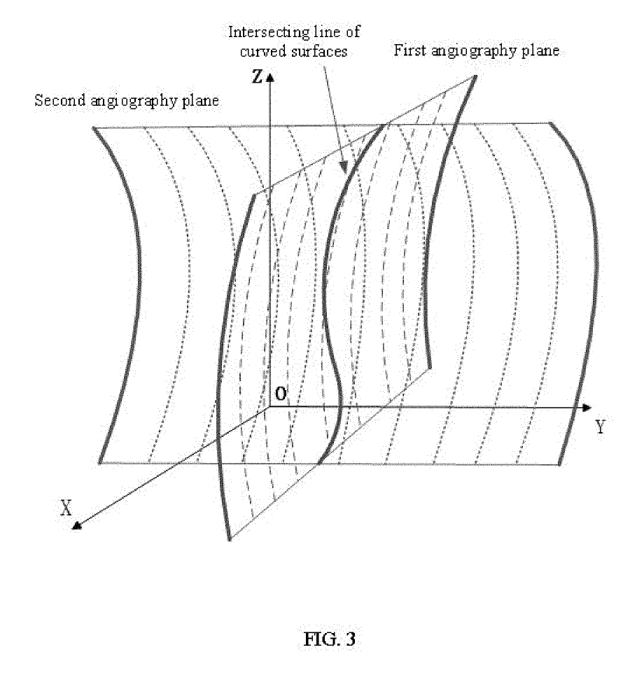

In the embodiment of the present disclosure, as shown in FIG. 3, the YOZ plane is the first angiography plane; the XOZ plane is the second angiography plane; two middle curved surfaces formed by dashed lines and solid lines are respectively the first curved surface perpendicularly intersected with the first angiography plane and the second curved surface perpendicularly intersected with the second angiography plane; and the intersecting line obtained by perpendicularly intersecting the first curved surface and the second curved surface is the three-dimensional guide wire. When the intersecting line of the two curved surfaces is solved, the two-dimensional guide wire of the coronary angiography image in the first or second angiography plane can be set as a reference target, and a Z coordinate of the two-dimensional guide wire of the coronary angiography image in the other angiography plane is compared with a Z coordinate of the reference target. When a difference value is within a preset threshold range, the Z coordinate of the reference target can be regarded as the intersecting line of the two curved surfaces.

Preferably, after the two-dimensional guide wires are translated, the two-dimensional guide wires are interpolated to generate B spline curve. The curved surfaces are built according to the B spline curve so that the curve is smoother.

In step S103, the intravascular ultrasound images per frame are arranged at equal intervals along the three-dimensional guide wire; and the intravascular ultrasound images are rotated to positions perpendicular to tangent vectors according to the tangent vectors in positions of the intravascular ultrasound images on the three-dimensional guide wire.

In the embodiment of the present disclosure, the intravascular ultrasound images per frame are located on the three-dimensional guide wire. The intravascular ultrasound images are sectional images of the entire vessels obtained by that an ultrasound probe is driven by a motor to move at a constant set speed along the guide wire; and the positions of the intravascular ultrasound images per frame on the three-dimensional guide wire can be obtained through calculation of a chord length method so that the intravascular ultrasound images per frame are arranged at equal intervals along the three-dimensional guide wire. As an example, when known parameters are frame numbers, frame quantity and total withdrawn length of the intravascular ultrasound images, the distances from the intravascular ultrasound images per frame to withdrawn points can be calculated, and then the positions of the intravascular ultrasound images per frame on the three-dimensional guide wire can be determined. When known parameters are frame quantity, frame rates and withdrawn rates of the intravascular ultrasound images, the total withdrawn length can be calculated, and then the spacing between adjacent intravascular ultrasound images can be calculated according to the quantity of the inner and outer membranes of the intravascular ultrasound images. Because the intravascular ultrasound images record the sections of the vessels, the intravascular ultrasound images also need to be rotated to be perpendicular to the tangent vectors in the corresponding positions of the three-dimensional guide wire.

Specifically, the intravascular ultrasound images per frame can be successively translated from the local coordinate system in which the three-dimensional guide wire is located to a preset world coordinate system (i.e., the coordinate system in which the coronary angiography image is located); and after translation, the positions of the intravascular ultrasound images on the three-dimensional guide wire coincide with an origin of the world coordinate system. The tangent vectors in the positions of the intravascular ultrasound images on the three-dimensional guide wire are acquired, and the intravascular ultrasound images are rotated according to angles formed by the tangent vectors with XOZ plane and YOZ of the world coordinate system.

In step S104, the intravascular ultrasound images in the corresponding positions of the tangent vectors are rotated at different angles in a vertical plane of the tangent vectors; the rotated intravascular ultrasound images are back projected to the coronary angiography image; and optimal orientation angles of the intravascular ultrasound images per frame are determined according to distances from the back projections of the intravascular ultrasound images and the vessel edge profile to the three-dimensional guide wire.

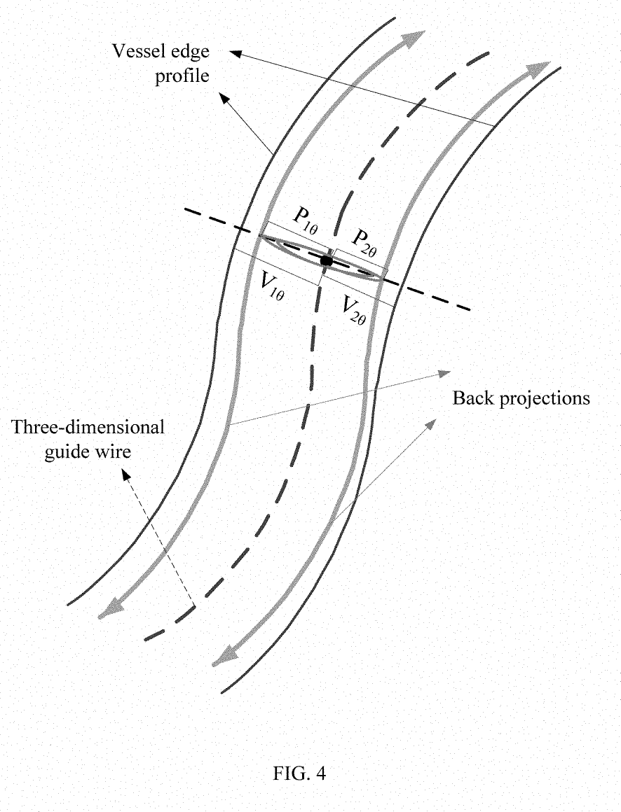

In the embodiment of the present disclosure, the tangent vectors are the tangent vectors in the positions of the intravascular ultrasound images on the three-dimensional guide wire. Because the vessel is an irregular column and the section of the vessel is not a standard circle, the intravascular ultrasound images need to be rotated at different angles in a vertical plane of the tangent vectors (for example, the intravascular ultrasound images can be set to rotate by 2 degrees each time, a total of 360 degrees). After rotation each time, the intravascular ultrasound images are back projected to the coronary angiography images in the first angiography plane and the second angiography plane, so as to find the optimal orientation angles of the intravascular ultrasound images per frame according to distances from the back projections of the intravascular ultrasound images and the vessel edge profile to the three-dimensional guide wire, thereby effectively reducing the three-dimensional reconstruction error of the vessel surface. As shown in FIG. 4, after the intravascular ultrasound images are rotated by θ angle, P_{1θ} and P_{2θ} are the distances from the back projections of the intravascular ultrasound images to the three-dimensional guide wire; V_{1θ} and V_{2θ} are the distances from the vessel edge profile to the three-dimensional guide wire.

In the embodiment of the present disclosure, reconstruction errors caused by rotating the intravascular ultrasound images at different angles are calculated according to the distances from the back projections of the intravascular ultrasound images and the vessel edge profile to the three-dimensional guide wire and based on a preset error accumulation formula, with the error accumulation formula being as follows:

$${e}_{θ} = {\sum }_{θ} ( \vert {P}_{1 θ} - {V}_{1 θ} \vert + \vert {P}_{2 θ} - {V}_{2 θ} \vert ) ,$$

wherein e_{θ} is a reconstruction error caused by rotating the intravascular ultrasound images at an angle of θ. Minimum reconstruction errors corresponding to the intravascular ultrasound images per frame are selected from all the reconstruction errors. The rotation angle corresponding to each minimum reconstruction error is the optimal orientation angle corresponding to the corresponding intravascular ultrasound image, thereby effectively reducing the calculation amount of orientation of the intravascular ultrasound images.

In step S105, the intravascular ultrasound images per frame are rotated to the corresponding optimal orientation angles; and surface reconstruction is conducted on vessels of the coronary angiography image and the intravascular ultrasound images according to a span difference between inner membranes and a span difference between outer membranes in the intravascular ultrasound images per frame on the three-dimensional guide wire.

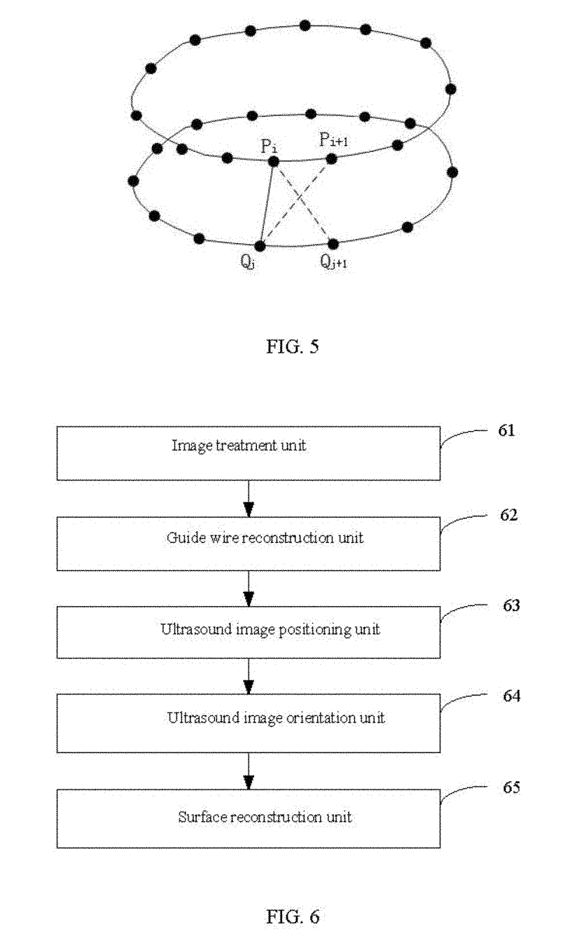

In the embodiment of the present disclosure, after the optimal orientation angles corresponding to the intravascular ultrasound images per frame are determined, the intravascular ultrasound images per frame are rotated to the corresponding optimal orientation angles, so that positioning and orientation of the intravascular ultrasound images on the coronary angiography image are completed. It can be known that the intravascular ultrasound images are composed of inner membranes and outer membranes, and after the inner membranes and the outer membranes are divided, inner membranes and outer membranes composed of discrete points can be obtained. Two layers of inner membranes are selected from the inner membranes of all the intravascular ultrasound images, and the two layers of selected inner membranes are set as target profile lines of an upper layer and a lower layer. As shown in FIG. 5, P₁, . . . , Pᵢ, Pᵢ₊₁, . . . is a vertex sequence on the upper-layer target profile line, and Q₁, . . . , Qᵢ, Qᵢ₊₁, . . . is a vertex sequence on the lower-layer target profile line. These data are the discrete points of the two layers of selected inner membranes. Similarly, two layers of outer membranes are selected from the outer membranes of all the intravascular ultrasound images.

In the embodiment of the present disclosure, the vessel surface can be reconstructed according to a preset minimum span method. Specifically, as shown in FIG. 5, when Pᵢ is closest to Qⱼ on the upper-layer target profile line, a triangular sheet that connects the upper-layer target profile line and the lower-layer target profile line is built on the basis of the span PⱼQᵢ. Namely, Qⱼ and Pᵢ are set as two vertexes of the triangular sheet; and the third vertex of the triangular sheet is determined based on a minimum span criterion: if the length of the span PᵢQⱼ₊₁ is smaller than the length of the span Pᵢ₊₁Qⱼ, then the third vertex of the triangular sheet is Qⱼ₊₁; three vertexes are connected to form the triangular sheet ΔQⱼPᵢQⱼ₊₁; otherwise, the third vertex of the triangular sheet is Pᵢ₊₁, and three vertexes are connected to form the triangular sheet ΔQⱼPᵢPᵢ₊₁. The triangular sheet is connected through continuous and circulative iteration until the triangular sheet encircles all profile vertexes by one circle. The above operation can be conducted in a hierarchical order of inner membranes or outer membranes and an order from the outer membranes to the inner membranes, and finally, the reconstruction of the vessel surface is completed.

In the embodiment of the present disclosure, the coronary angiography image is pretreated, thereby effectively reducing the adverse effect of the image noise on the three-dimensional reconstruction accuracy of the vessels. The vessel edge profile can be extracted from the pretreated coronary angiography image, and the two-dimensional guide wires in the coronary angiography image can be extracted through a Hessian matrix, so that the accurate positions of the two-dimensional guide wires can be still found when the vessel is changed suddenly. The inner membranes and the outer membranes are divided for the intravascular ultrasound images, and the three-dimensional guide wire is generated according to the two-dimensional guide wires, and the first angiography plane and the second angiography plane of the coronary angiography image, thereby effectively reducing errors generated by the three-dimensional guide wire due to no calibration of part of parameters or parameter deviation in the angiography device. After the three-dimensional guide wire is determined, positions and directions of the intravascular ultrasound images on the three-dimensional guide wire are determined. During orientation, the calculation amount is effectively reduced through back projection, and finally the vessel surface is reconstructed, thereby realizing the integration of the coronary angiography and the intravascular ultrasound images, so that shapes and structures, and intraluminal lesion information of the vessels can be examined simultaneously and efficiency and accuracy of three-dimensional reconstruction of the coronary vessels are effectively enhanced.

### Embodiment 2

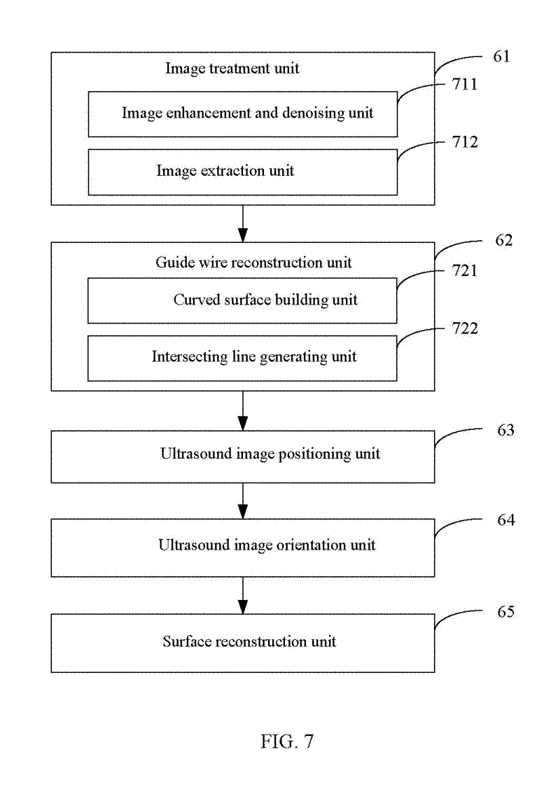

FIG. 6 shows a structure of a three-dimensional reconstruction apparatus for coronary vessels according to embodiment 2 of the present disclosure. For the convenience of explanation, only the part related to the embodiment of the present disclosure is shown, including: an image treatment unit 61 for pretreating an inputted coronary angiography image, extracting a vessel edge profile and two-dimensional guide wires from the pretreated coronary angiography image, and dividing inner and outer membranes for inputted associated intravascular ultrasound images; a guide wire reconstruction unit 62 for translating the two-dimensional guide wires located in a first angiography plane and a second angiography plane in the coronary angiography image to the same starting point; building perpendicularly intersected curved surfaces according to the translated two-dimensional guide wires; and setting an intersecting line of the perpendicularly intersected curved surfaces as a three-dimensional guide wire; an ultrasound image positioning unit 63 for arranging the intravascular ultrasound images per frame at equal intervals along the three-dimensional guide wire and rotating the intravascular ultrasound images to positions perpendicular to tangent vectors according to the tangent vectors in positions of the intravascular ultrasound images on the three-dimensional guide wire; an ultrasound image orientation unit 64 for rotating the intravascular ultrasound images in the corresponding positions of the tangent vectors at different angles in a vertical plane of the tangent vectors, back projecting the rotated intravascular ultrasound images to the coronary angiography image, and determining optimal orientation angles of the intravascular ultrasound images per frame according to distances from the back projections of the intravascular ultrasound images and the vessel edge profile to the three-dimensional guide wire; and a surface reconstruction unit 65 for rotating the intravascular ultrasound images per frame to the corresponding optimal orientation angles, and conducting surface reconstruction on vessels of the coronary angiography image and the intravascular ultrasound images according to a span difference between inner membranes and a span difference between outer membranes in the intravascular ultrasound images per frame on the three-dimensional guide wire.

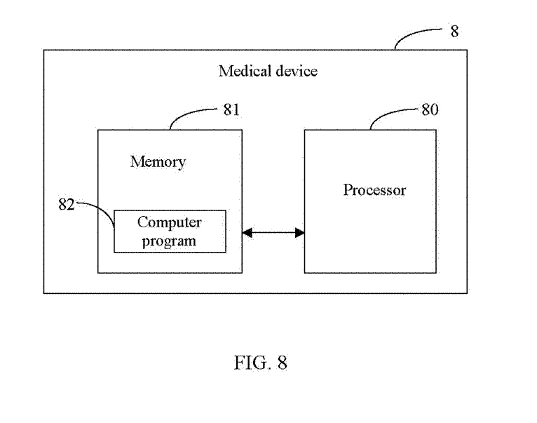

Preferably, as shown in FIG. 7, the image treatment unit 61 includes: an image enhancement and denoising unit 711 for conducting contrast enhancement on the coronary angiography image and smoothing noise on the coronary angiography image; and an image extraction unit 712 for extracting the vessel edge profile on the coronary angiography image and extracting the two-dimensional guide wires of the vessels in the coronary angiography image according to a preset Hessian matrix extraction mode.

Preferably, the guide wire reconstruction unit 62 includes: a curved surface building unit 721 for respectively building a first curved surface perpendicularly intersected with the first angiography plane and a second curved surface perpendicularly intersected with the second angiography plane according to the translated two-dimensional guide wires; and an intersecting line generating unit 722 for perpendicularly intersecting the first curved surface and the second curved surface to generate the intersecting line, and setting the intersecting line as a three-dimensional guide wire.

In the embodiment of the present disclosure, the coronary angiography image is pretreated, thereby effectively reducing the adverse effect of the image noise on the three-dimensional reconstruction accuracy of the vessels. The vessel edge profile can be extracted from the pretreated coronary angiography image, and the two-dimensional guide wires in the coronary angiography image can be extracted through a Hessian matrix, so that the accurate positions of the two-dimensional guide wires can be still found when the vessel is changed suddenly. The inner membranes and the outer membranes are divided for the intravascular ultrasound images, and the three-dimensional guide wire is generated according to the two-dimensional guide wires, and the first angiography plane and the second angiography plane of the coronary angiography image, thereby effectively reducing errors generated by the three-dimensional guide wire due to no calibration of part of parameters or parameter deviation in the angiography device. After the three-dimensional guide wire is determined, positions and directions of the intravascular ultrasound images on the three-dimensional guide wire are determined. During orientation, the calculation amount is effectively reduced through back projection, and finally the vessel surface is reconstructed, thereby realizing the integration of the coronary angiography and the intravascular ultrasound images, so that shapes and structures, and intraluminal lesion information of the vessels can be examined simultaneously and efficiency and accuracy of three-dimensional reconstruction of the coronary vessels are effectively enhanced. Specific implementation contents of each unit in the embodiment of the present disclosure can be seen from the description of corresponding steps in embodiment 1, and will not be repeated.

In the embodiment of the present disclosure, each unit of the three-dimensional reconstruction apparatus for coronary vessels can be realized through corresponding hardware or software units. All units can be individual software or hardware units, and can also be integrated into one software or hardware unit, not used to limit the present disclosure herein.

### Embodiment 3

FIG. 8 shows a structure of a medical device according to embodiment 3 of the present disclosure. For the convenience of explanation, only the part related to the embodiment of the present disclosure is shown.

The medical device 8 in the embodiment of the present disclosure includes a processor 80, a memory 81, and a computer program 82 stored in the memory 81 and executed on the processor 80. The processor 80, when executing the computer program 82, realizes the steps in the above method embodiment, such as steps S101 to S105 shown in FIG. 1. Or, the processor 80, when executing the computer program 82, realizes the function of each unit in the above apparatus embodiment, such as the functions of units 61-65 shown in FIG. 6.

In the embodiment of the present disclosure, the coronary angiography image is pretreated; the vessel edge profile is extracted; the two-dimensional guide wires are extracted; and the inner membranes and the outer membranes are divided for the intravascular ultrasound images. The coronary angiography images located in the preset first angiography plane and the preset second angiography plane are translated, so that the two-dimensional guide wire in the coronary angiography image of the first angiography plane has the same starting point as the two-dimensional guide wire in the coronary angiography image of the second angiography plane. After translation, the perpendicularly intersected curved surfaces are built according to the two-dimensional guide wires; and an intersecting line of the curved surfaces is set as a three-dimensional guide wire. The intravascular ultrasound images per frame are arranged at equal intervals along the three-dimensional guide wire; and the intravascular ultrasound images are rotated so that the intravascular ultrasound images are perpendicular to tangent vectors in corresponding positions on the three-dimensional guide wire. The corresponding intravascular ultrasound images are rotated at different angles in a vertical plane of the tangent vectors; the rotated intravascular ultrasound images are back projected to the coronary angiography image; and optimal orientation angles of the intravascular ultrasound images per frame are determined according to distances from the back projections and the vessel edge profile to the three-dimensional guide wire. The vessel surface is reconstructed according to the span difference between the inner membranes and the span difference between the outer membranes in the intravascular ultrasound images per frame on the three-dimensional guide wire, thereby realizing the integration of the coronary angiography and the intravascular ultrasound images, so that shapes and structures, and intraluminal lesion information of the vessels can be examined simultaneously. In addition, the present disclosure effectively reduces the influence of image noise on vascular reconstruction due to respiration of patients, effectively solves the influence caused by lack of parameters or incomplete parameter calibration in the angiography device, and effectively enhances efficiency and accuracy of three-dimensional reconstruction of the coronary vessels.

### Embodiment 4

In the embodiment of the present disclosure, a computer readable storage medium is provided. The computer readable storage medium stores the computer program, wherein the computer program, when executed by the processor, realizes the steps in the above method embodiment, such as steps S101 to S105 shown in FIG. 1. Alternatively, the computer program, when executed by the processor, realizes the function of each unit in the above apparatus embodiment, such as the functions of units 61-65 shown in FIG. 6.

In the embodiment of the present disclosure, the coronary angiography image is pretreated; the vessel edge profile is extracted; the two-dimensional guide wires are extracted; and the inner membranes and the outer membranes are divided for the intravascular ultrasound images. The coronary angiography images located in the preset first angiography plane and the preset second angiography plane are translated, so that the two-dimensional guide wire in the coronary angiography image of the first angiography plane has the same starting point as the two-dimensional guide wire in the coronary angiography image of the second angiography plane. After translation, the perpendicularly intersected curved surfaces are built according to the two-dimensional guide wires; and an intersecting line of the curved surfaces is set as a three-dimensional guide wire. The intravascular ultrasound images per frame are arranged at equal intervals along the three-dimensional guide wire; and the intravascular ultrasound images are rotated so that the intravascular ultrasound images are perpendicular to tangent vectors in corresponding positions on the three-dimensional guide wire. The corresponding intravascular ultrasound images are rotated at different angles in a vertical plane of the tangent vectors; the rotated intravascular ultrasound images are back projected to the coronary angiography image; and optimal orientation angles of the intravascular ultrasound images per frame are determined according to distances from the back projections and the vessel edge profile to the three-dimensional guide wire. The vessel surface is reconstructed according to the span difference between the inner membranes and the span difference between the outer membranes in the intravascular ultrasound images per frame on the three-dimensional guide wire, thereby realizing the integration of the coronary angiography and the intravascular ultrasound images, so that shapes and structures, and intraluminal lesion information of the vessels can be examined simultaneously. In addition, the present disclosure effectively reduces the influence of image noise on vascular reconstruction due to respiration of patients, effectively solves the influence caused by lack of parameters or incomplete parameter calibration in the angiography device, and effectively enhances efficiency and accuracy of three-dimensional reconstruction of the coronary vessels.

The computer readable storage medium in the embodiment of the present disclosure may include any entity or apparatus or recording medium capable of carrying computer program codes, such as ROM/RAM, disk, optical disc, flash memory and other memories.

The above only describes preferred embodiments of the present disclosure, not used to limit the present disclosure. Any change, equivalent replacement and improvement made within the spirit and principle of the present disclosure shall be included in the protection scope of appended claims.

We claim:

1. A three-dimensional reconstruction method for coronary vessels, comprising the following steps:
pretreating an inputted coronary angiography image, extracting a vessel edge profile and two-dimensional guide wires from the pretreated coronary angiography image, and dividing inner and outer membranes for inputted associated intravascular ultrasound images;
translating the two-dimensional guide wires located in a first angiography plane and a second angiography plane in the coronary angiography image to a same starting point, building perpendicularly intersected curved surfaces according to the translated two-dimensional guide wires, and setting an intersecting line of the perpendicularly intersected curved surfaces as a three-dimensional guide wire;
arranging the intravascular ultrasound images per frame at equal intervals along the three-dimensional guide wire, and rotating the intravascular ultrasound images to positions perpendicular to tangent vectors according to the tangent vectors in positions of the intravascular ultrasound images on the three-dimensional guide wire;
rotating the intravascular ultrasound images in the corresponding positions of the tangent vectors at different angles in a vertical plane of the tangent vectors, back projecting the rotated intravascular ultrasound images to the coronary angiography image, and determining optimal orientation angles of the intravascular ultrasound images per frame according to distances from the back projections of the intravascular ultrasound images and the vessel edge profile to the three-dimensional guide wire; and
rotating the intravascular ultrasound images per frame to the corresponding optimal orientation angles, and conducting surface reconstruction on vessels of the coronary angiography image and the intravascular ultrasound images according to a span difference between inner membranes and a span difference between outer membranes in the intravascular ultrasound images per frame on the three-dimensional guide wire.

2. The method according to claim 1, wherein the step of pretreating an inputted coronary angiography image and extracting a vessel edge profile and two-dimensional guide wires from the pretreated coronary angiography image comprises:
conducting contrast enhancement on the coronary angiography image and smoothing noise on the coronary angiography image; and
extracting the vessel edge profile on the coronary angiography image and extracting the two-dimensional guide wires in the coronary angiography image according to a preset Hessian matrix extraction mode.

3. The method according to claim 1, wherein the step of building perpendicularly intersected curved surfaces according to the translated two-dimensional guide wires and setting an intersecting line of the perpendicularly intersected curved surfaces as a three-dimensional guide wire comprises:
respectively building a first curved surface perpendicularly intersected with the first angiography plane and a second curved surface perpendicularly intersected with the second angiography plane according to the translated two-dimensional guide wires; and
perpendicularly intersecting the first curved surface and the second curved surface to generate the intersecting line; and setting the intersecting line as a three-dimensional guide wire.

4. The method according to claim 1, wherein the step of arranging the intravascular ultrasound images per frame at equal intervals along the three-dimensional guide wire and rotating the intravascular ultrasound images to positions perpendicular to tangent vectors according to the tangent vectors in positions of the intravascular ultrasound images on the three-dimensional guide wire comprises:
calculating corresponding positions of the intravascular ultrasound images per frame on the three-dimensional guide wire and arranging the intravascular ultrasound images per frame at equal intervals along the three-dimensional guide wire according to the corresponding positions; and
translating the intravascular ultrasound images per frame to a preset world coordinate system; rotating the intravascular ultrasound images per frame according to directions of the tangent vectors corresponding to the intravascular ultrasound images in the world coordinate system so that the planes of the intravascular ultrasound images per frame are perpendicular to the corresponding tangent vectors; and translating the intravascular ultrasound images per frame to the coordinate system of the three-dimensional guide wire.

5. The method according to claim 1, wherein the step of determining optimal orientation angles of the intravascular ultrasound images per frame according to distances from the back projections of the intravascular ultrasound images and the vessel edge profile to the three-dimensional guide wire comprises:
calculating reconstruction errors caused by rotating the intravascular ultrasound images per frame on the vertical plane at different angles according to the distances from the back projections of the intravascular ultrasound images and the vessel edge profile to the three-dimensional guide wire and based on a preset error accumulation formula, with the error accumulation formula being as follows:
$${e}_{θ} = {\sum }_{θ} ( \vert {P}_{1 θ} - {V}_{1 θ} \vert + \vert {P}_{2 θ} - {V}_{2 θ} \vert ) ,$$
wherein θ is an angle at which the intravascular ultrasound images are rotated on the vertical plane; e_{θ} is a reconstruction error caused by rotating the intravascular ultrasound images at an angle of θ; P_{1θ} and P_{2θ} are the distances from the back projections of the intravascular ultrasound images to the three-dimensional guide wire; V_{1θ} and V_{2θ} are the distances from the vessel edge profile to the three-dimensional guide wire; and
acquiring minimum reconstruction errors corresponding to the intravascular ultrasound images per frame; and correspondingly setting the rotation angle corresponding to each minimum reconstruction error as the optimal orientation angles of the intravascular ultrasound images per frame.

6. A three-dimensional reconstruction apparatus for coronary vessels, comprising:
an image treatment unit for pretreating an inputted coronary angiography image, extracting a vessel edge profile and two-dimensional guide wires from the pretreated coronary angiography image, and dividing inner and outer membranes for inputted associated intravascular ultrasound images;
a guide wire reconstruction unit for translating the two-dimensional guide wires located in a first angiography plane and a second angiography plane in the coronary angiography image to a same starting point, building perpendicularly intersected curved surfaces according to the translated two-dimensional guide wires, and setting an intersecting line of the perpendicularly intersected curved surfaces as a three-dimensional guide wire;
an ultrasound image positioning unit for arranging the intravascular ultrasound images per frame at equal intervals along the three-dimensional guide wire and rotating the intravascular ultrasound images to positions perpendicular to tangent vectors according to the tangent vectors in positions of the intravascular ultrasound images on the three-dimensional guide wire;
an ultrasound image orientation unit for rotating the intravascular ultrasound images in the corresponding positions of the tangent vectors at different angles in a vertical plane of the tangent vectors, back projecting the rotated intravascular ultrasound images to the coronary angiography image, and determining optimal orientation angles of the intravascular ultrasound images per frame according to distances from the back projections of the intravascular ultrasound images and the vessel edge profile to the three-dimensional guide wire; and
a surface reconstruction unit for rotating the intravascular ultrasound images per frame to the corresponding optimal orientation angles, and conducting surface reconstruction on vessels of the coronary angiography image and the intravascular ultrasound images according to a span difference between inner membranes and a span difference between outer membranes in the intravascular ultrasound images per frame on the three-dimensional guide wire.

7. The apparatus according to claim 6, wherein the image treatment unit comprises:
an image enhancement and denoising unit for conducting contrast enhancement on the coronary angiography image and smoothing noise on the coronary angiography image; and
an image extraction unit for extracting the vessel edge profile on the coronary angiography image and extracting the two-dimensional guide wires of vessels in the coronary angiography image according to a preset Hessian matrix extraction mode.

8. The apparatus according to claim 6, wherein the guide wire reconstruction unit comprises:
a curved surface building unit for respectively building a first curved surface perpendicularly intersected with the first angiography plane and a second curved surface perpendicularly intersected with the second angiography plane according to the translated two-dimensional guide wires; and
an intersecting line generating unit for perpendicularly intersecting the first curved surface and the second curved surface to generate the intersecting line, and setting the intersecting line as a three-dimensional guide wire.

9. A medical device, comprising a memory, a processor and a computer program stored in the memory and executed on the processor, wherein the processor, when executing the computer program, realizes the method of claim 1.

10. A computer readable storage medium for storing a computer program, wherein the computer program, when executed by a processor, realizes the method of claim 1.
